(19) United States
(12) Patent Application Publication
(10) Pub. No.: US 2024/0050375 A1
(43) Pub. Date: Feb. 15, 2024

(54) MASKING THE TASTE OF ISOSORBIDE

(71) Applicant: ROQUETTE FRERES, Lestrem (FR)

(72) Inventors: Grégory LE BIHAN, ANNEZIN (FR); Sébastien CROQUET, MERVILLE (FR)

(21) Appl. No.: 17/754,498

(22) PCT Filed: Oct. 7, 2020
(86) PCT No.: PCT/FR2020/051766

§ 371 Date: Apr. 4, 2022

(30) Foreign Application Priority Data
Oct. 7, 2019 (FR) 19 11102

(51) Int. Cl. A61K 9/16; A61K 31/34

(52) U.S. Cl. CPC A61K 9/1652; A61K 31/34; A61K 9/1682

(57) ABSTRACT

The present technology relates to masking the taste of isosorbide, in particular its bitterness. The technology developed herein proposes a new dosage form allowing the taste of isosorbide to be improved, as well as a method for producing this dosage form.

### TECHNICAL FIELD

The present technology relates to masking the taste of isosorbide, in particular its bitterness. The technology developed herein thus proposes a new dosage form allowing the taste of isosorbide to be improved, as well as a process for preparing this dosage form.

### BACKGROUND ART

The majority of active substances used in pharmaceutical forms intended for oral administration are characterized by an unpleasant taste. However, nowadays taste is becoming an essential criterion for making the administration of a medicament acceptable, and thereby improving treatment compliance. Therefore, masking the taste is a real challenge for drug developers, particularly for medicaments intended for pediatric and geriatric populations, for whom oral administration is always more problematic. There are many taste-masking techniques available for drug formulations, based on various approaches. There is no one definitive method, with the choice of a technique predominantly depending on the active substance to be masked, on the pharmaceutical form, and on the cost of the technology in question. Within the list of techniques, a distinction is made between methods which are simple to implement, such as adding flavorings or sweeteners, but which are often inadequate, necessitating the use of more complex techniques such as the use of cyclodextrins or ion-exchange resins.

Isosorbide is an osmotic diuretic used in several countries in the treatment of Ménière's disease. This active ingredient, which needs to be ingested in large amounts, is known for its extremely unpleasant taste, in particular its strong bitterness. Typically, a dose of isosorbide consists of the oral absorption of 40 ml sticks containing 70% isosorbide (i.e. 28 g of isosorbide administered per dose). However, to date, no method has been found which makes it possible to effectively mask the taste of isosorbide, in particular considering the concentrations and amounts ingested.

### AIM OF THE INVENTION

Therefore, the aim of the present invention is to provide an effective means for masking the taste of isosorbide, in particular when this active ingredient is administered in large amounts and/or at high concentrations.

### PRESENTATION OF THE INVENTION

Herein, the inventors propose a dosage form which makes it possible to solve this problem. This oral dosage form is in the form of gelled beads of isosorbide, preferably using alginate as gelling agent.

As will be clear on reading the examples below, this method makes it possible to administer large amounts of isosorbide without the patient perceiving the unpleasant taste. The gelled beads obtained in this way may contain more than 80% by dry weight of isosorbide.

Still on reading the examples below, it is observed that this method advantageously succeeds where conventional taste-masking techniques, such as the use of sweeteners, flavorings, or cyclodextrins, have failed.

Moreover, the dosage form proposed by the present invention is simple to implement, since it does not require the use of a large number of materials.

With isosorbide being a small, highly soluble molecule, it is surprising to observe that the bitterness is not perceived, especially considering the amounts and concentrations administered. Indeed, it would be expected that the isosorbide would very rapidly diffuse out of the beads and into the saliva of the oral cavity, where its bitterness would be detected by the taste buds, and therefore perceived by the panel of tasters.

For example, document WO2006/001344 relates to a gelled preparation based on alginate isosorbide and other gelling constituents. Such a composition is not suitable for producing beads. Moreover, this document teaches that taste masking is optimized by adding cocoa powder. Document US2010/120712 relates to a composition comprising these two ingredients, said composition being in the form of extruded granules or powders. Compositions in bead form are not described.

The use of gelled alginate beads for masking the taste of active ingredients is mentioned in the prior art. However, it has never been proposed to apply this technology to isosorbide in order to mask the taste thereof. There is nothing in the prior art to suggest that this technology is applicable to isosorbide, in particular considering that significant amounts of isosorbide are administered.

For example, document WO 00/06122 describes pharmaceutical compositions for oral administration, comprising particles of active ingredient and a gelling agent. The particles of active ingredient may optionally be in the form of alginate beads (example 12). The particles of active ingredient obtained are mixed with a gelling agent and other substances in order to obtain the final dosage form. During administration, this solid form is added to water in order to obtain a gelled composition, and the unpleasant taste of the active ingredient is masked. This document does not mention isosorbide, and the pharmaceutical compositions ultimately ingested comprise very low amounts of active ingredient.

### SUMMARY OF THE INVENTION

Thus, the invention firstly relates to gelled beads, particularly for oral administration, comprising isosorbide, the isosorbide content being at least 50% by dry weight of isosorbide relative to the total weight of said gelled beads.

Secondly, the invention relates to a pharmaceutical composition comprising or consisting of the gelled beads of the invention.

Thirdly, the invention relates to a process for preparing the gelled beads of the invention, said process comprising the following steps:
step (a): preparing a solution comprising isosorbide and one or more gelling agent(s);
step (b): preparing a gelation solution;
step (c) adding the solution prepared in step (a) in a dropwise manner to the gelation solution prepared in step (b);
step (d) collecting the gelled beads of isosorbide obtained in this way.

Fourthly, the invention relates to a method for masking the unpleasant taste of isosorbide, consisting in putting said isosorbide in the form of gelled beads.

### DESCRIPTION OF EMBODIMENTS

The characteristics disclosed in the following paragraphs may optionally be implemented. They may be implemented independently of one another or in combination with one another.

The invention firstly relates to gelled beads, particularly for oral administration, comprising isosorbide, the isosorbide content being at least 50% by dry weight of isosorbide relative to the total weight of said gelled beads. Preferably, this content by dry weight of isosorbide is at least 60%, preferably at least 70%, preferably at least 75%, more preferably still at least 80%. This content by dry weight of isosorbide is generally less than or equal to 95%, or even less than or equal to 90%, or even less than or equal to 85%.

This content by dry weight of isosorbide, relative to the total weight of said gelled beads, can be determined by the person skilled in the art, for example by gas chromatography with flame ionization detection and internal calibration, preferably using methyl α-D-glucopyranoside as internal standard, for example according to a protocol described in the examples below.

The gelled beads of the invention typically comprise one or more gelling agent(s). Gelling agents capable of rapidly gelling at ambient temperature and in the presence of water are particularly useful here. These are preferably polymers capable of undergoing crosslinking gelation in the presence of polyvalent metal ions.

The gelling agents include, inter alia, agents capable of gelling when they are used in combination. Examples of gelling agents are alginates, pectates, carrageenans and gelatin. Use will preferably not be made of gelatin, especially due to the fact that it is a product of animal origin. Use will preferably at least be made of alginate as gelling agent. When several gelling agents are present, the proportion by dry weight of alginate is preferably at least 20% relative to the total dry weight of gelling agents, preferably at least 30%, preferably at least 40%, preferably at least 50%, preferably at least 60%, preferably at least 70%, preferably at least 80%, preferably at least 90%. More preferably still, the alginate is the only gelling agent in the gelled beads.

Generally, these gelling agents are in the form of salts, for example sodium, magnesium, potassium salts, preferably sodium salts. Sodium alginate is particularly preferred.

Preferably, the gelling agents used have a Brookfield viscosity, as measured at 1% at 20° C., of less than or equal to 2000 cps, preferably less than or equal to 1000 cps, preferably less than or equal to 800 cps, preferably less than or equal to 500 cps, preferably less than or equal to 400 cps, preferably less than or equal to 300 cps, preferably less than or equal to 250 cps, preferably less than or equal to 200 cps. Preferably, this Brookfield viscosity is greater than or equal to 10 cps, preferably greater than or equal to 20 cps, preferably greater than or equal to 30 cps, preferably greater than or equal to 35 cps, preferably greater than or equal to 50 cps, preferably greater than or equal to 80 cps, preferably greater than or equal to 100 cps. This viscosity is for example selected in a range extending from 35 to 65 cps, or from 100 to 200 cps, or from 300 to 400 cps.

Preferably, the content by dry weight of gelling agents relative to the total dry weight of the gelled beads (dry/dry), in particular the alginate content, is at least 0.1%, preferably at least 0.3%, preferably at least 0.5%, preferably at least 0.7%, preferably at least 1.0%, preferably at least 1.2%, preferably at least 1.4%. This dry/dry weight content of gelling agents or more particularly of alginate is preferably less than or equal to 5.0%, preferably less than or equal to 4.0%, preferably less than or equal to 3.0%, preferably less than or equal to 2.0%, preferably less than or equal to 1.8%, preferably less than or equal to 1.6%.

Preferably, the gelled beads of the invention have a dry weight ratio of isosorbide/gelling agents at least equal to 40, preferably at least equal to 50, preferably at least equal to 55, preferably at least equal to 60. This ratio is preferably less than or equal to 200, preferably less than or equal to 150, preferably less than or equal to 140, preferably less than or equal to 130, preferably less than 120, preferably less than or equal to 100, preferably less than or equal to 80, preferably less than or equal to 75.

Preferably, when the alginate is used, the gelled beads of the invention have a dry weight ratio of isosorbide/alginate at least equal to 40, preferably at least equal to 50, preferably at least equal to 55, preferably at least equal to 60. This ratio is preferably less than or equal to 200, preferably less than or equal to 150, preferably less than or equal to 140, preferably less than or equal to 130, preferably less than 120, preferably less than or equal to 100, preferably less than or equal to 80, preferably less than or equal to 75.

The gelled beads of the present invention comprise a solvent, preferably water, even more preferentially demineralized water. Preferably, the solvent content of the gelled beads is less than or equal to 30% by weight relative to the total weight of gelled beads, preferably less than or equal to 25%, preferably less than or equal to 20%, for example less than or equal to 18%. This solvent content is generally greater than or equal to 1%, or even greater than or equal to 5%, or even greater than or equal to 10%, for example greater than or equal to 13%, 14%, or 15%.

The gelled beads may contain ions, generally in trace amounts, depending on the process employed for the preparation of said beads.

Moreover, the gelled beads may contain substances other than those listed above, as long as this does not disrupt the desired properties, especially regarding the taste quality of the gelled beads, and/or the stability thereof, and/or the pharmacological activity of the isosorbide. Other such substances are, for example: flavorings, sweeteners; in particular intense sweeteners; encapsulating agents such as cyclodextrins; active ingredients other than isosorbide; compounds targeting the bioavailability of the active ingredients of the beads, in particular aiming to modify the bioavailability of isosorbide.

Preferably, the gelled beads comprise less than 30% by dry weight of other substances, relative to the total weight of gelled beads, preferably less than 20%, preferably less than 10%, preferably less than 5%, preferably less than 1%, preferably 0%. Indeed, it has been shown, in the examples below, that the other substances are not necessary for solving the problem posed herein.

Most preferentially, the gelled beads are devoid of other substances. In particular, this means that the gelled beads in this case consist solely of isosorbide, of gelling agents, and of a solvent which is preferably water, more preferentially demineralized water. In one advantageous embodiment, the gelled beads consist solely of isosorbide, of alginate, and of a solvent which is preferably water, more preferentially demineralized water.

Preferably, the gelled beads have a mean diameter of less than or equal to 5.0 mm, preferably less than or equal to 3.0 mm, preferably less than or equal to 2.0 mm, preferably less than or equal to 1.5 mm. This mean diameter is generally greater than or equal to 0.1 mm, or even greater than or equal to 0.2 mm, or even greater than or equal to 0.3 mm, or even greater than or equal to 0.4 mm, or even greater than or equal to 0.5 mm.

Preferably, the beads are insoluble in water at a temperature of 20° C.

The invention also relates to a pharmaceutical composition comprising the gelled beads of the invention. The isosorbide acts as active ingredient in this pharmaceutical composition.

“Pharmaceutical composition” is intended to mean a composition in the final dosage form thereof, intended to be administered to a patient.

This pharmaceutical composition may also only consist of the gelled beads of the invention. Thus, the gelled beads may indeed be administered as is. Alternatively, the gelled beads may be administered with other substances, for example in the form of a suspension of the gelled beads in a syrup.

Preferably, the pharmaceutical composition of the invention contains an amount of isosorbide per dose of at least 10 g, preferably at least 20 g, preferably at least 25 g, for example 28 g.

Preferably, the pharmaceutical composition of the invention is intended to be administered 1, 2 or 3 times daily, preferably 3 times daily.

Preferably, the pharmaceutical composition of the invention contains an isosorbide content of at least 20% by dry weight relative to the total weight of said pharmaceutical composition, preferably at least 30% by dry weight, preferably at least 40% by dry weight, preferably at least 50% by dry weight, preferably at least 60% by dry weight, preferably at least 70% by dry weight, preferably at least 75% by dry weight, more preferably still at least 80% by dry weight. This content by dry weight of isosorbide is generally less than or equal to 95%, or even less than or equal to 90%, or even less than or equal to 85%.

Preferably, the pharmaceutical composition according to the invention is for use as medicament, in particular for treating Ménière's disease. Another subject matter of the invention relates to a treatment method, in particular for Ménière's disease, comprising the administration of a pharmaceutical composition of the invention. In other words, this is a composition for use thereof in the treatment of Ménière's disease. Moreover, it is also a question of the use of the composition according to the invention for producing a medicament intended for a therapeutic use in the treatment of Ménière's disease. Preferably, the patient to be treated is an individual suffering from Ménière's disease.

Another subject matter of the present invention also relates to a process for preparing gelled beads according to the invention, said process comprising the following steps:
step (a): preparing a solution comprising isosorbide and one or more gelling agent(s);
step (b): preparing a gelation solution;
step (c) adding the solution prepared in step (a) in a dropwise manner to the gelation solution prepared in step (b);
step (d) collecting the gelled beads of isosorbide obtained in this way.

Preferably, for the preparation of the solution of step (a), the gelling agent is first dissolved in a solvent, which is preferably water, more preferentially demineralized water, and the isosorbide is added subsequently.

Preferably, the solution of step (a) has an isosorbide content selected in a range extending from 30 to 80% by weight relative to the total weight of said solution. Preferably, this content is at least equal to 40%, preferably at least equal to 45%, preferably at least equal to 50%, preferably at least equal to 55%. Preferably, this content is at most equal to 80%, preferably at most equal to 75%, preferably at most equal to 70%, preferably at most equal to 65%. This content is for example equal to 50% or to 60%.

Preferably, the solution of step (a) has a content of gelling agents, in particular an alginate content, selected in a range extending from 0.1 to 5.0% by weight relative to the total weight of said solution. Preferably, this content is at least equal to 0.2%, preferably at least equal to 0.3%, preferably at least equal to 0.4%, preferably at least equal to 0.5%. Preferably, this content is at most equal to 4.5%, preferably at most equal to 4.0%, preferably at most equal to 3.5%, preferably at most equal to 3.0%, preferably at most equal to 2.5%, preferably at most equal to 2.0%, preferably at most equal to 1.5%, preferably at most equal to 1.0%.

Preferably, the amounts of isosorbide and of gelling agents of the solution of step (a) are selected such that the isosorbide/gelling agents weight ratio is at least equal to 40, preferably at least equal to 50, preferably at least equal to 60. This ratio is preferably less than or equal to 200, preferably less than or equal to 150, preferably less than or equal to 140, preferably less than or equal to 110, preferably less than or equal to 100.

Preferably, when the alginate is used, the amounts of isosorbide and of alginate of the solution of step (a) are selected such that the isosorbide/alginate weight ratio is at least equal to 40, preferably at least equal to 50, preferably at least equal to 60. This ratio is preferably less than or equal to 200, preferably less than or equal to 150, preferably less than or equal to 140, preferably less than or equal to 110, preferably less than or equal to 100.

Preferably, the total amount of solids in the solution of step (a) is selected in a range extending from 30 to 80% by weight relative to the total weight of said solution. Preferably, this solids amount is selected in a range extending from 50 to 70%, preferably from 60 to 65%.

Preferably, the gelation solution of step (b) comprises one or more polyvalent metal ion(s). Indeed, it is recalled that the gelling agents of the invention are preferably polymers capable of undergoing crosslinking gelation in the presence of polyvalent metal ions. Alternatively, and depending on the gelling agents selected, the gelation solution may be a solution having a temperature lower than that of the solution of step (a), such that gelation occurs by cooling.

These polyvalent metal ions are for example selected from calcium, aluminum, iron, copper or zinc ions, or a mixture thereof. Calcium ions are particularly preferred. These calcium ions may preferably be in the form of inorganic salts such as calcium chloride, calcium sulfate, calcium monohydrogen phosphate, calcium carbonate. They may also be in the form of organic salts such as calcium lactate, calcium gluconate, calcium citrate. It is preferably a water-soluble salt. Most preferentially, the polyvalent metal ions of use in the invention comprise at least calcium chloride. More preferably still, calcium chloride is the only polyvalent metal ion used.

If the polyvalent metal ions are in the form of water-insoluble salts (as is the case for example for calcium carbonate), it will be necessary to add an acid to dissolve the salt, in particular an organic acid such as citric acid, adipic acid, glucono-delta-lactone acid, etc. This is why preference is given to using water-soluble calcium salts, in particular calcium chloride.

Preferably, the gelation solution of step (b) has a content of polyvalent metal ions, in particular a calcium chloride content, selected in a range extending from 1 to 20% by dry weight relative to the total weight of said solution. Preferably, this content is at least equal to 3%, preferably at least equal to 5%, preferably at least equal to 7%, preferably at least equal to 9%. Preferably, this content is at most equal to 18%, preferably at most equal to 16%, preferably at most equal to 14%, preferably at most equal to 12%, preferably at most equal to 11%. This content is for example equal to 10%.

In order to carry out step (c), it is possible to pump the solution of step (a), contained in a storage unit, in order to convey it to the gelation solution, contained in a different storage unit. The pump used will typically depend on the viscosity of the solution of step (a) containing the gelling agent(s). The dropwise addition of the solution prepared in step (a) to the gelation solution prepared in step (b) enables the instant formation of beads.

Preferably, the process for preparing gelled beads further comprises, between steps (c) and (d), a step of washing the gelled beads, preferably in demineralized water. This step is typically carried out in order to remove salts optionally used in the process, for example calcium chloride, from the product.

The process for preparing gelled beads preferably comprises a step of drying the gelled beads, between steps (c) and (d). This step will preferably be after the washing step, if said washing step is carried out. This drying can be carried out in an oven. Nevertheless, on the industrial scale, techniques using the action of a stream of heated air applied to the moving beads are preferred. One example is drying in a fluidized air bed.

In addition to increasing the stability of the gelled beads, the drying also has the effect of reducing the diameter of the beads. Thus, in order to control for the desired diameter, in addition to choosing a suitable system for forming the drops to be gelled, it will be necessary to take into account the effect of this heating step.

Preferably, before drying, the gelled beads contain a liquid core. Indeed, in this case, the drying step will advantageously be able to concentrate the isosorbide in the beads.

Preferably, after drying, the gelled beads do not contain a liquid core.

The present invention also relates to gelled beads, especially for oral administration, which are able to be obtained by, or obtained according to, the process for preparing gelled beads of the invention. The preferred embodiments of these gelled beads are as described above in the description relating to the gelled beads.

Another subject matter of the invention relates to a method for masking the unpleasant taste of isosorbide, consisting in putting said isosorbide in the form of gelled beads.

“Unpleasant taste” is conventionally intended to mean the savors and/or flavors perceived as such by a set of individuals. In the case in point, referring to isosorbide, this is typically its bitterness.

Preferably, the gelled beads make it possible to statistically significantly reduce the bitterness of isosorbide compared to an isosorbide solution having the same concentration and amount of isosorbide, said bitterness being detected by an electronic tongue equipped with bitterness sensors having a lipid membrane, for example using equipment of the type Insent® Electronic Taste Sensing System TS-5000Z (Atsugi-Chi, Japan), equipped for example with bitterness sensors SB2ACO (Bitterness 1, cationic substances), SB2AN0 (Bitterness 2, cationic substances) and SB2C00 (Bitterness 3, anionic substances).

Preferably, the gelled beads make it possible to statistically significantly improve the taste of isosorbide compared to an isosorbide solution having the same concentration and amount of isosorbide, said improvement being determined using a panel of tasters.

Alternatively, instead of comparing to an isosorbide solution of the same concentration, it will also be possible to compared to a 40 ml isosorbide solution comprising 28 g of isosorbide, which represents a reference dosage for treating Ménière's disease.

Preferably, the gelled beads make it possible to totally mask the unpleasant taste of isosorbide, and in particular its bitterness.

Preferably, the gelled beads are as described above.

Preferably, the isosorbide is put in the form of gelled beads by the process for preparing gelled beads of the invention as described above.

In the present description, it is understood that an amount by “dry weight” refers to an amount by weight of anhydrous substance. On the contrary, and unless indicated otherwise, an amount simply expressed by “weight” (typically in the part of the description relating to preparation processes) refers to amounts of substances referred to as “commercial”, i.e. to amounts of generally pulverulent product, used as is. These contents by weight therefore include the water optionally present intrinsically in these commercial powders. In this regard, it will be noted that isosorbide and the salts thereof typically and preferably contain 0% by weight of water, and that the gelling agents, in particular the alginate, comprise at most 15% by weight of water.

The drawings and the following description substantially contain elements which are certain in nature. They will therefore be able not only to serve to better understand the present invention, but also contribute to defining the invention, where appropriate.

### EXAMPLES

### Materials

Demineralized water; Isosorbide (Isosorbide C PHARMA, ROQUETTE, batch E2366), xylitol (XYLISORB® P90, ROQUETTE, batch E302Y); maltitol (SWEETPEARL® P90, ROQUETTE, batch EMM29); amylose-rich pea maltodextrin (KLEPTOSE® Linecaps, ROQUETTE, batch E4118); hydroxypropyl-beta-cyclodextrin (KLEPTOSED HPB, ROQUETTE, batch E0262); yellow pectin (LOUIS FRANCOIS, batch 342CS), a compound (“coprocessed compound”) of microcrystalline cellulose (MCC) and of carboxymethylcellulose (CMC) (Tabulose 591 F, ROQUETTE, batch 165004073); soluble hydrolyzed hydroxypropyl pea starch (LYCOAT® RS 720, ROQUETTE, batch E001R), aspartame (AJINOMOTO); sodium saccharin (SIGMA); sucralose (NIUTANG); citric acid (SIGMA); low-viscosity sodium alginate (Brookfield viscosity of 35-56 cps at 1.0%, 20° C.) (sodium alginate IL6G, AGI); medium-viscosity sodium alginate (Brookfield viscosity of 100-200 cps at 1.0%, 20° C.) (sodium alginate I1G80, AGI); high-viscosity sodium alginate (Brookfield viscosity of 300-400 cps at 1.0%, 20° C.) (sodium alginate I3G80, AGI); calcium chloride (SIGMA); flavorings (MANE): Banana/Bitter masking/Blackcurrant/Cherry/Herbal/Lemon/Mint/Orange Peach/Strawberry/Tutti frutti/chocolate/caramel.

### Example 1: Masking the Taste of Isosorbide using Intense Sweeteners (Liquid Medium)

Various liquid formulations were tested in order to assess the efficacy of the intense sweeteners in masking the taste of isosorbide. The formulations used are presented in the following tables 1 and 2, and the percentages are expressed by weight relative to the total weight of the formulation.

TABLE 1

| | Formu- | Formu- | Formu- | Formu- |
|---|---|---|---|---|
| Reference | lation 1 | lation 2 | lation 3 | lation 4 |
| Demineralized | 29.945 | 29.912 | 27.945 | 27.912 |
| water (%) | | | | |
| Isosorbide (%) | 70 | 70 | 70 | 70 |
| Aspartame (%) | 0.05 | 0.08 | 0.05 | 0.08 |
| Sodium | 0.005 | 0.008 | 0.005 | 0.008 |
| saccharin (%) | | | | |
| Sucralose (%) | — | — | — | — |
| Maltitol (%) | — | — | 1 | 1 |
| Xylitol (%) | — | — | 1 | 1 |

TABLE 2

| | Formu- | Formu- | Formu- | Formu- |
|---|---|---|---|---|
| Reference | lation 5 | lation 6 | lation 7 | lation 8 |
| Demineralized | 29.975 | 29.95 | 27.975 | 27.95 |
| water (%) | | | | |
| Isosorbide (%) | 70 | 70 | 70 | 70 |
| Aspartame (%) | — | — | — | — |
| Sodium | — | — | — | — |
| saccharin (%) | | | | |
| Sucralose (%) | 0.025 | 0.05 | 0.025 | 0.05 |
| Maltitol (%) | — | — | 1 | 1 |
| Xylitol (%) | — | — | 1 | 1 |

The solutions thus prepared were administered orally to a panel of 5 tasters.

The use of aspartame and sodium saccharin did not make it possible to reduce the bitter sensation caused by the isosorbide. Substituting aspartame and sodium saccharin for sucralose also did not make it possible to reduce the bitter sensation caused by the isosorbide, even when the concentrations of sucralose were increased. The addition of maltitol and xylitol in the formulations also did not make it possible to improve the taste of the preparations.

### Example 2: Masking the Taste of Isosorbide using Encapsulation Techniques(Liquid Medium)

Various liquid formulations were tested in order to assess the efficacy of techniques of encapsulation by complexation in masking the taste of isosorbide. The formulations used are presented in the following tables 3 and 4, and the percentages are expressed by weight relative to the total weight of the formulation.

TABLE 3

| Reference | Formulation 9 | Formulation 10 | Formulation 11 |
|---|---|---|---|
| Demineralized water | 29 | 28 | 25 |
| (%) | | | |
| Isosorbide (%) | 70 | 70 | 70 |
| Amylose-rich pea | 1 | 2 | 5 |
| maltodextrin (%) | | | |
| Hydroxypropyl-beta- | — | — | — |
| cyclodextrin (%) | | | |

TABLE 4

| Reference | Formulation 12 | Formulation 13 | Formulation 14 |
|---|---|---|---|
| Demineralized water | 29 | 28 | 25 |
| (%) | | | |
| Isosorbide (%) | 70 | 70 | 70 |
| Amylose-rich pea | — | — | — |
| maltodextrin (%) | | | |
| Hydroxypropyl-beta- | 1 | 2 | 5 |
| cyclodextrin (%) | | | |

The solutions thus prepared were administered orally to a panel of 5 tasters.

Regardless of the concentrations of hydroxypropyl-beta-cyclodextrin and of pea maltodextrin used, the impact on the bitterness was limited. A taste of burnt caramel was perceived by the panel for all of formulations 9 to 14, but the bitterness was not eliminated.

### Example 3: Masking the Taste of Isosorbide using Texturing Techniques (Gelled Medium)

Various formulations in gel form were tested in order to assess the efficacy of texturing techniques in masking the taste of isosorbide. The formulations used are presented in the following tables 5 and 6, and the percentages are expressed by weight relative to the total weight of the formulation. For the preparation of these formulations, the isosorbide was milled beforehand (IKA mill) in order to facilitate dissolution thereof.

TABLE 5

| | Formu- | Formu- | Formu- | Formu- | Formu- |
|---|---|---|---|---|---|
| | lation | lation | lation | lation | lation |
| Reference | 15 | 16 | 17 | 18 | 19 |
| Demineralized | 29 | 25 | 20 | 25 | 20 |
| water (%) | | | | | |
| Isosorbide (%) | 70 | 70 | 70 | 70 | 70 |
| MCC and CMC | 1 | 5 | 10 | — | — |
| compound (%) | | | | | |
| Soluble | — | — | — | 5 | 10 |
| hydrolyzed | | | | | |
| hydroxypropyl | | | | | |
| pea starch (%) | | | | | |
| Yellow | — | — | — | — | — |
| pectin (%) | | | | | |
| Maltitol (%) | — | — | — | — | — |
| Citric acid (%) | — | — | — | — | — |
| Aspartame (%) | — | — | — | — | — |
| Sodium | — | — | — | — | — |
| saccharin (%) | | | | | |
| Sucralose (%) | — | — | — | — | — |

TABLE 6

| Reference | Formulation 20 | Formulation 21 | Formulation 22 | Formulation 23 | Formulation 24 |
|---|---|---|---|---|---|
| Demineralized | 27 | 26.95 | 27 | 26.945 | 26.912 |
| water (%) | | | | | |
| Isosorbide (%) | 70 | 70 | 70 | 70 | 70 |
| MCC and CMC | — | — | — | — | — |
| compound (%) | | | | | |
| Soluble | — | — | — | — | — |
| hydrolyzed | | | | | |
| hydroxypropyl pea | | | | | |
| starch (%) | | | | | |
| Yellow pectin (%) | 1 | 1 | 1 | 1 | 1 |
| Maltitol (%) | 1 | 1 | 1 | 1 | 1 |
| Citric acid (%) | 1 (10% | 1 (10% | 1 (10% | 1 (10% | 1 (10% |
| | solution) | solution) | solution) | solution) | solution) |
| Aspartame (%) | — | — | — | 0.05 | 0.08 |
| Sodium saccharin | — | — | — | 0.005 | 0.008 |
| (%) | | | | | |
| Sucralose (%) | — | 0.05 | 0.08 | — | — |

The gels thus prepared were administered orally to a panel of 5 tasters.

The use of an MCC/CMC compound and of soluble hydrolyzed hydroxypropyl pea starch made it possible to obtain a somewhat viscous gel, depending on the concentration used. The use of yellow pectin made it possible to obtain a better texture.

The formulations with the MCC/CMC compound and pectin had a limited impact on the bitterness of the isosorbide, even when intense sweeteners were added to the formulation. The formulations based on soluble hydrolyzed hydroxypropyl pea starch did not make it possible to improve the taste of the isosorbide.

### Example 4: Masking the Taste of Isosorbide using Texturing Techniques (Gelled Medium) and Using Flavorings

In this example, the inventors attempted to improve the gels of example 3 which only had a limited impact on the bitterness of the isosorbide. To this end, gels with flavorings added were tested. The formulations used are presented in the following tables 7 and 8, and the percentages are expressed by weight relative to the total weight of the formulation. For the preparation of these formulations, the isosorbide was milled beforehand (IKA mill) in order to facilitate dissolution thereof. All the flavorings listed in the “Materials” section above were tested, that is the following flavorings: Banana/Bitter masking/Blackcurrant/Cherry/Herbal/Lemon/Mint/Orange Peach/Strawberry/Tutti frutti/chocolate/caramel.

TABLE 7

| | Formu- | Formu- | Formu- | Formu- |
|---|---|---|---|---|
| Reference | lation 25 | lation 26 | lation 27 | lation 28 |
| Demineralized water (%) | 26.85 | 26.75 | 26.82 | 26.72 |
| Isosorbide (%) | 70 | 70 | 70 | 70 |
| Yellow pectin (%) | 1 | 1 | 1 | 1 |
| Maltitol (%) | 1 | 1 | 1 | 1 |
| Citric acid (%) | 1 (10% | 1 (10% | 1 (10% | 1 (10% |
| | solution) | solution) | solution) | solution) |
| Aspartame (%) | — | — | — | — |
| Sodium saccharin (%) | — | — | — | — |
| Sucralose (%) | 0.05 | 0.05 | 0.08 | 0.08 |
| Flavoring (%) | 0.1 | 0.2 | 0.1 | 0.2 |

TABLE 8

| | Formu- | Formu- | Formu- | Formu- |
|---|---|---|---|---|
| Reference | lation 29 | lation 30 | lation 31 | lation 32 |
| Demineralized water (%) | 26.845 | 26.745 | 26.812 | 26.712 |
| Isosorbide (%) | 70 | 70 | 70 | 70 |
| Yellow pectin (%) | 1 | 1 | 1 | 1 |
| Maltitol (%) | 1 | 1 | 1 | 1 |
| Citric acid (%) | 1 (10% | 1 (10% | 1 (10% | 1 (10% |
| | solution) | solution) | solution) | solution) |
| Aspartame (%) | 0.05 | 0.05 | 0.08 | 0.08 |
| Sodium saccharin (%) | 0.005 | 0.005 | 0.008 | 0.008 |
| Sucralose (%) | — | — | — | — |
| Flavoring (%) | 0.1 | 0.2 | 0.1 | 0.2 |

The flavored gels thus prepared were administered orally to a panel of 5 tasters.

Yet again, the results were not conclusive, regardless of the flavoring tested and regardless of the concentration thereof. For all the tests, strong bitterness continued to be detected by the panel.

### Example 5: Masking the Taste of Isosorbide by Forming Gelled Beads

Various formulations in the form of gelled beads were tested in order to assess the efficacy of this technology in masking the taste of isosorbide.

The solutions [A] presented in the following tables 9 and 10 were first prepared (the percentages are expressed by weight relative to the total weight of the formulation).

TABLE 9

| | Formu- | Formu- | Formu- |
|---|---|---|---|
| Reference | lation 33 | lation 34 | lation 35 |
| Demineralized water (%) | 49.5 | 39.5 | 29.5 |
| Isosorbide (%) | 50.0 | 60.0 | 70.0 |
| Sodium alginate (%) | 0.5 | 0.5 | 0.5 |
| Isosorbide/alginate | 100 | 120 | 140 |
| weight ratio | | | |

TABLE 10

| Reference | Formulation 36 | Formulation 37 | Formulation 38 |
|---|---|---|---|
| Demineralized water | 49.2 | 49.0 | 39.0 |
| (%) | | | |
| Isosorbide (%) | 50.0 | 50.0 | 60.0 |
| Sodium alginate (%) | 0.8 | 1.0 | 1.0 |
| Isosorbide/alginate | 63 | 50 | 60 |
| weight ratio | | | |

The sodium alginate was dispersed in demineralized water and everything was mixed using a high-shear mixer (of POLYTRON type). The isosorbide was added after having been milled in order to facilitate the dissolution thereof.

A gelation solution [B] was subsequently prepared, consisting of 90% by weight of demineralized water, and 10% by weight of calcium chloride.

For each solution [A], the following was carried out: the solution [A] was taken off using a pipette, and then added dropwise to the solution [B], in order to instantly obtain gelled beads. The beads thus formed were removed and rinsed in demineralized water in a beaker in order to eliminate the residual calcium chloride.

The beads thus obtained had a diameter of approximately 2-3 mm. They were subsequently dried in an oven, and then had a diameter of approximately 1 mm.

In order to determine if the compounds of solution [A] had completely passed into gelled bead form, a test was performed which consisted in introducing a blue stain into solution [A]. No staining of solution [B] was observed during the formation of the beads, thus indicating that the compounds of solution [A] were substantially contained in these beads.

Three different sodium alginates were tested for formulations 33 to 37: a low-viscosity alginate (35-65 cps), a medium-viscosity alginate (100-200 cps), and a high-viscosity alginate (300-400 cps). All three alginates made it possible to obtain satisfactory beads. However, the inventors noted that the medium-viscosity alginate represented an optimum, especially in terms of the drying efficiency of the gelled beads. This medium-viscosity alginate was used for formulation 38.

The gelled beads of isosorbide thus obtained thoroughly masked the bitterness of isosorbide. Compared to formulation 33, when the isosorbide concentration was increased in solution (formulations 34 and 35), the beads were weaker. When the alginate concentration was increased in the solutions containing 50% isosorbide (formulations 36 and 37), beads were obtained which did not have a liquid core (the beads were totally gelled). At this stage it is recalled that the presence of a liquid core is advantageous, because drying will then make it possible to effectively concentrate the isosorbide. Ultimately, the best beads were obtained from a solution [A] containing 60% isosorbide, 39% demineralized water and 1.0% sodium alginate (formulation 38 with an isosorbide/alginate ratio=60; solids content=61%), followed by the beads obtained from a solution [A] containing 50% isosorbide, 49.5% demineralized water and 0.5% sodium alginate (formulation 33 with an isosorbide/alginate ratio=100; solids content=50.5%).

The beads obtained from formulation 38 were analyzed by gas chromatography with flame ionization detection and internal calibration in order to determine their content by dry weight of isosorbide.

The capillary column used was 30 meters long, had an internal diameter of 0.32 millimeters, and a film thickness of 1 micron.

The operating conditions were as follows: column temperature 140-250° C., at a rate of 3° C./minute, then up to 300° C. at a rate of 10° C./min, injector temperature 300° C.; detector temperature 300° C.; vector gas helium; constant flow rate 1.7 ml/min, split injection mode; flow rate of split 80 ml/minute, hydrogen flow rate 30 ml/minute, air flow rate 400 ml/minute, volume injected 1 microliter

Between 100 and 150 mg of gelled beads and 30 mg of internal standard (methyl α-D-glucopyranoside) were placed in a 100 ml beaker. 10 ml of 10% sodium dodecyl sulfate and 50 ml of reverse osmosis-purified water were added, and everything was placed under stirring in order to dissolve the gelled beads.

1 ml of solution, to which 1 ml of pyridine was added, was deposited in a 2 ml dish with screw-on lid. Everything was evaporated to dryness under a stream of nitrogen. The residue was taken up again in 1 ml of pyridine and 0.5 ml of BSTFA, and placed under stirring or sonication in order to detach the deposit. Everything was left dry in the bath, temperature controlled to 70° C. for 30 minutes, before injecting 1 microliter.

The isosorbide content is expressed in g dry per 100 g of gelled beads, and is given by the following equation:

$$\% isosorbide = \frac{Si}{Se} \times \frac{Pe}{P} \times \frac{100}{Ki} \qquad [ Math . 1 ]$$

with:
Si=surface area of the isosorbide;
Se=surface area of the internal standard peak;
Pe=weight of internal standard introduced into beaker (in mg);
P=weight of beads weighed (in mg)
Ki=response factor of the isosorbide (approximately 0.8 in the analysis conditions used here).

Two samples, which did not differ in the drying method used, were tested. The sample dried in a turbine had a content by dry weight of isosorbide relative to the total weight of gelled beads of 81.7±0.9% (mean produced over 9 measurements). The sample dried in an oven had a content by dry weight of isosorbide relative to the total weight of gelled beads of 83.6±1.0% (mean over 6 measurements).

1. Gelled beads comprising isosorbide, the isosorbide content being at least 50% by dry weight of isosorbide relative to the total weight of said gelled beads.

2. The gelled beads according to claim 1, wherein the isosorbide content is at least 60% by dry weight of isosorbide relative to the total weight of said gelled beads.

3. The gelled beads according to claim 1, comprising one or more gelling agent(s).

4. The gelled beads according to claim 3, wherein one or more gelling agent(s) comprises alginate.

5. The gelled beads according to claim 3, wherein the content by dry weight of gelling agents relative to the total dry weight of the gelled beads is at least 0.1%, and less than or equal to 5.0%.

6. The gelled beads according to claim 3, wherein they the gelled beads have a dry weight ratio of isosorbide/gelling agents at least equal to 40.

7. The gelled beads according to claim 1, wherein they the gelled beads have a water content of less than or equal to 30% by weight relative to the total weight of gelled beads.

8. The gelled beads according to claim 1, wherein they the gelled beads have a mean diameter of less than or equal to 5.0 mm.

9. A pharmaceutical composition comprising or consisting of the gelled beads as defined in claim 1.

10. The pharmaceutical composition according to claim 9, for use thereof as medicament.

11. A process for preparing gelled beads as defined in claim 1, said process comprising the following steps:
step (a): preparing a solution comprising isosorbide and one or more gelling agent(s);
step (b): preparing a gelation solution;
step (c) adding the solution prepared in step (a) in a dropwise manner to the gelation solution prepared in step (b);
step (d) collecting the gelled beads of isosorbide obtained in this way.

12. The process for preparing gelled beads according to claim 11, wherein the gelation solution of step (b) comprises one or more polyvalent metal ion(s).

13. The process for preparing gelled beads according to claim 12, wherein said polyvalent metal ion(s) are selected from calcium, aluminum, iron, copper or zinc ions or a mixture thereof.

14. The process for preparing gelled beads according to claim 11, wherein it further comprises a step of drying the gelled beads.

15. A method for masking the unpleasant taste of isosorbide, consisting of putting said isosorbide in the form of gelled beads.
